(19) United States
(12) Patent Application Publication
(10) Pub. No.: US 2011/0142949 A1
(43) Pub. Date: Jun. 16, 2011

(54) CHONDROITINASE TREATMENT METHOD FOR DEMYELINATION-RELATED CONDITIONS AND DISEASES

(75) Inventors: Donna J. Osterhout, East Syracuse, NY (US); Dennis Stelzner, Syracuse, NY (US)

(73) Assignee: The Research Foundation of State University of New York, Albany, NY (US)

(21) Appl. No.: 12/906,399

(22) Filed: Oct. 18, 2010

Related U.S. Application Data
(60) Provisional application No. 61/252,365, filed on Oct. 16, 2009.

(51) Int. Cl. A61K 38/43; A61K 9/14; A61P 25/00

(52) U.S. Cl. 424/497; 424/94.1; 424/490

(57) ABSTRACT

A method of treating a mammal suffering from a demyelinating disease or condition, the method comprising administering chondroitinase to said mammal at a site where demyelination has occurred.

### CROSS-REFERENCE TO RELATED APPLICATIONS

This application claims the benefit of U.S. Provisional Application No. 61/252,365 filed on Oct. 16, 2009.

### FIELD OF THE INVENTION

The present invention is directed to the use of chondroitinase in treating a disease or condition of the nervous system.

### BACKGROUND OF THE INVENTION

There are a number of diseases and conditions that exhibit demyelination as a primary characteristic. In demyelination, the protective myelin sheath that ordinarily surrounds neurons becomes damaged. This damage of the myelin sheath causes an impairment in signal conduction, and ultimately, an impairment in, for example, sensation, cognition, and motor skills.

Of particular significance in the class of demyelination diseases are the idiopathic inflammatory demyelinating diseases (IIDDs), also known as borderline forms of multiple sclerosis (MS). Multiple sclerosis is a particularly debilitating type of demyelinating disease in which the body's immune system attacks the central nervous system (i.e., brain and spinal cord) to cause demyelination, which typically results in a range of acute conditions associated with a lack of neurologic function. In addition to these diseases, there are some conditions that include demyelination. Most notable in this regard is spinal cord injury.

Though therapies, including medications, have been used to manage and ameliorate the symptoms of demyelinating diseases and conditions, such as MS and spinal cord injury, there is no known cure for these types of diseases and conditions. More particularly, there is no known method for repairing or reversing the demyelination (i.e., remyelination) in these diseases and conditions. Without a method for remyelination, a demyelinating disease or condition will generally progress even when the disease or condition is being managed by a therapeutic treatment.

Accordingly, there is a significant need for a means to treat demyelination diseases and conditions by a remyelination therapy. Such a therapy would treat the very source of such debilitating conditions as MS and spinal cord injury, and thereby, provide a future for sufferers of such diseases in which the disease does not worsen but instead progressively abates or reaches a leveling point that can be maintained. It would be further advantageous and beneficial if the remyelination therapy is substantially devoid of acute side effects.

### SUMMARY OF THE INVENTION

The invention is directed to a method of treating a mammal suffering from a demyelinating disease or condition by administering chondroitinase (i.e., “cABC enzyme” or “cABC”) to the mammal at a site where demyelination has occurred. The invention is also directed to compositions containing chondroitinase that are suitable for administering to a mammal for the purpose of remyelinating demyelinated tissue.

The administration of chondroitinase has surprisingly been found to promote, initiate, or catalyze the remyelination of nerve tissue that contains myelin in a damaged or depleted condition, or wherein the nerve tissue is completely demyelinated. The method advantageously treats the very cause of such debilitating diseases and conditions as MS and spinal cord injury. The remyelination therapy described herein is also advantageously substantially devoid of acute side effects.

### BRIEF DESCRIPTION OF THE DRAWINGS

FIGS. 1A,B. Micrographs of cABC-treated cells fixed with 4% paraformaldehyde 96 hours post-plating.

FIGS. 2A-2D. FIG. 2A: drawing depicting a preferred methodology for inducing injury in animals and, thereafter, treatment with cABC. FIG. 2B: drawing depicting a preferred methodology wherein, to determine the effectiveness of cABC treatment following SCI, 9 photomicrographs were taken in the general areas indicated. FIG. 2C: drawing depicting a preferred methodology for determining the geographic distribution of OPCs. FIG. 2D: micrograph showing that only cells that were double stained for both PDGFRα and Olig1 and displayed a clear cell morphology were counted in the analysis (scale bar=10 μm).

FIGS. 3A-3F. Graphs showing that the use of cABC resulted in a doubling in the number of PDGFRα/Olig1 labeled OPCs.

### DETAILED DESCRIPTION OF THE INVENTION

In one aspect, the invention is directed to a method of treating a mammal suffering from a demyelinating disease or condition by administering chondroitinase to the mammal at a site where demyelination has occurred. The demyelinating disease or condition considered herein can be any such disease or condition in which demyelination occurs to any extent. Some examples of demyelinating diseases and conditions include those encompassed within IIDDs, such as MS, as well as, for example, spinal cord injury (e.g., acute or chronic), transverse myelitis, progressive multifocal leukoencephalopathy, optic neuritis, Devic's disease, leukodystrophies.

The cABC enzyme employed by the present invention may be either the natural cABC enzyme produced by and isolated from Proteus vulgaris, or a recombinant cABC enzyme produced by and isolated from other expression systems. Specifically, the DNA sequence that encodes the amino acid sequence of the cABC enzyme can be cloned from Proteus vulgaris, incorporated into one or more vectors (e.g., plasmids, phages, cosmids, phagemids, and viruses), and then recombinantly expressed in either a prokaryotic or eukaryotic host organism (e.g., E. coli bacteria or yeast). For example, Vikas Prabhakar and colleagues described a process for cloning the cABC-encoding DNA sequence from Proteus vulgaris and recombinantly expressing it in E. coli to form recombinant cABC I enzyme (Prabhakar et al., “Chondroitinase ABC I from Proteus vulgaris: cloning, recombinant expression and active site identification,” BIOCHEM. J., Vol. 386, pp. 103-112 (2005)).

Further, the present invention may utilize a functional derivative of the cABC enzyme. As used herein, the term “functional derivative” of the cABC enzyme is a compound that possesses a biological activity (either functional or structural) that is substantially similar to that of the cABC enzyme. The term “functional derivative” is intended to include the biologically active fragments, variants, analogs and homologues, or the chemical derivatives of the cABC enzyme. The term “fragment” is meant to refer to any polypeptide subset of the cABC enzyme. The term “variant” is meant to refer to a molecule substantially similar in structure and function to either the entire cABC enzyme or to a fragment thereof. The term “analog” refers to a molecule substantially similar in function to either the entire cABC enzyme or to a fragment thereof. For example, the functional derivative of the cABC enzyme may preferably contain the middle domain of the cABC enzyme where the active site of the cABC enzyme is located, and more preferably the highly conserved residues His501, Tyr508, Arg560 and Glu653 that are critical and essential for the cABC enzymatic activity.

The chondroitinase can be administered in any manner that permits intact chondroitinase to contact demyelinated tissue or tissue in contact with demyelinated tissue. Preferably, the chondroitinase is administered by injection of a sterile solution containing chondroitinase and a pharmaceutically acceptable carrier (i.e., an aqueous-based non-toxic vehicle) in which chondroitinase is dissolved. Alternatively, a solid or gel biodegradable material or carrier structure impregnated with chondroitinase can be implanted at the site of demyelination.

As used herein, a “pharmaceutically acceptable carrier” includes any of the solvents, dispersion media, antibacterial and antifungal agents, isotonic and absorption delaying agents, and the like commonly used in the pharmaceutical arts and that are physiologically compatible. Preferably, the carrier is suitable for injection into a mammal. The pharmaceutical solution can include one or more excipients commonly used in the art. Some excipients include, for example, pharmaceutically acceptable stabilizers, thickeners, and disintegrants.

The mammal to be treated is typically human. However, other mammals, such as monkeys, dogs, and cats can be treated by the methods described herein.

One or more additional agents can also be administered simultaneously with chondroitinase, i.e., either by injection of a solution containing chondroitinase and the one or more additional agents or by administration of spheres (i.e., microspheres or nanospheres) encapsulating chondroitinase together with the one or more additional agents. The agents can function, for example, as adjuvants, auxiliary agents, promoting agents, or therapeutic agents. In a particular embodiment, the additional agent is a protein or proteoglycan.

A class of proteins particularly considered herein is the class of neurotrophins (i.e., neurotrophic factors). Some examples of neurotrophins include nerve growth factor (NGF), brain-derived neurotrophic factor (BDNF), neurotrophin-3 (NT-3), neurotrophin-4 (NT-4), ciliary neurotrophic factor (CNTF), and glial cell line-derived growth factor (GDNF).

Some other classes of proteins particularly considered herein are the cytokines, growth factor hormones, antibodies, and carrier proteins. Some examples of cytokines particularly considered herein include leukemia inhibitory factor (LIF), interleukin 6 (IL6), interleukin 11 (IL11), and cardiotrophin 1. Some examples of growth factor hormones particularly considered herein include interferon α (IFNα), interferon β (IFNβ), and tumor necrosis factor (TNF). A particular class of antibodies considered herein includes antibodies that block the inhibitory activity of certain proteoglycans (such as NG2 proteoglycan). Some examples of carrier proteins particularly considered herein include bovine serum albumin (BSA), keyhole limpet hemocyanin (KLH), ovalbumin (OVA), fetal bovine serum (FBS), thyroglobulin (THY), and human serum albumin (HSA). Such carrier proteins can enhance the stability of cABC enzyme in vivo.

In a particular embodiment, the chondroitinase being administered is contained within or on biodegradable carrier structures (e.g., spheres). The carrier structures (i.e., biodegradable encapsulants) contain chondroitinase, a suitable vehicle, and optionally, one or more additional agents.

Preferably, the biodegradable encapsulant is composed of one or more biocompatible and biodegradable polymeric materials. The polymeric material can be a homopolymer or copolymer. A copolymer can be derived from two, three, or more chemically distinct types of monomer units. A copolymer can also be an alternating, block, graft, or random copolymer. The term “biocompatible” denotes that the polymeric material is compatible with a living tissue or a living organism by not being toxic or injurious and by not causing an immunological reaction. The term “biodegradable” denotes that the polymeric material will degrade over time by the action of, for example, hydrolytic mechanisms (e.g., enzymatic mechanisms) in the body of a subject.

In a particular embodiment, the polymeric materials of the biodegradable encapsulant are biodegradable polyesters. Some examples of biodegradable polyesters include those derived from α-hydroxycarboxylic acids, hydroxydicarboxylic acids, poly-α-cyanoacrylic acids and esters, amino acids, and combinations thereof. Some examples of α-hydroxycarboxylic acids include glycolic acid, lactic acid, 2-hydroxybutyric acid, leucic acid, and vanillic acid. Some examples of hydroxydicarboxylic acids include malic acid and tartronic acid. Some examples of hydroxytricarboxylic acids include citric acid and isocitric acid. Some examples of cyanoacrylic esters include methyl-α-cyanoacrylate, ethyl-α-cyanoacrylate, and butyl-α-cyanoacrylate. Some examples of particularly suitable amino acids include glycine, alanine, and γ-benzyl-L-glutamate.

Preferred biodegradable polymers employed as the encapsulant are homopolymers or copolymers derived from one or more kinds of α-hydroxycarboxylic acids, particularly glycolic acid, lactic acid, and 2-hydroxybutyric acid. Particularly preferred copolymers are derived from a mixture (i.e., two or more kinds) of α-hydroxycarboxylic acids.

The encapsulant can also be composed of a mixture of two or more biodegradable polymers. For example, in one embodiment, the encapsulant is composed of a mixture of two or more α-hydroxycarboxylic acid homopolymers. In another embodiment, the encapsulant is composed of a mixture of two or more α-hydroxycarboxylic acid copolymers. In another embodiment, the encapsulant is composed of a mixture of one or more α-hydroxycarboxylic acid homopolymers and one or more α-hydroxycarboxylic acid copolymers.

When the α-hydroxycarboxylic acids are chiral compounds, they may be of D, L, or mixed (i.e., D and L) configuration. In a particular embodiment, the ratio of the D/L configuration (mol %) is in the range from about 75/25 to about 25/75. In another embodiment, the polymer is derived from a α-hydroxycarboxylic acid wherein the ratio of the D/L configuration (mol %) is in the range from about 60/40 to about 30/70.

In different embodiments, the biodegradable polymer or copolymer preferably has a weight average molecular weight of about, at least, or no more than 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, or 200,000, or a particular range bounded by any two of these values.

In a particular embodiment, the α-hydroxycarboxylic acid polymer is a lactic acid polymer (i.e., a polylactic acid or PLA polymer) or copolymer. The lactic acid copolymer includes any copolymer derived from lactic acid in combination with any of the α-hydroxycarboxylic acid units described above. Some particular lactic acid copolymers include those containing lactic acid units in combination with 2-hydroxybutyric acid and/or glycolic acid units.

The polylactic acid may be either D-configuration or L-configuration, or a mixture thereof. Preferably, the D/L configuration ratio (mol %) is from about 75/25 to about 20/80. More preferred is a polylactic acid wherein the ratio of the D/L configuration (mol %) is in the range of about 60/40 to about 25/75. The polylactic acid can be produced by methods known in the art, including, for example, dehydrative polycondensation in the absence of a catalyst or by dehydrative polycondensation in the presence of an inorganic solid acid catalyst.

In a particular embodiment, the copolymer is a poly(lactic acid-co-glycolic acid) (i.e., PLGA) copolymer. In different embodiments, the lactic acid/glycolic acid molar compositional ratio in the PLGA copolymer is preferably from about 95/5 to about 40/60, or about 90/10 to about 45/55, or about 85/15 to 50/50.

In another embodiment, the encapsulant is a glycolic acid copolymer (e.g., lactic acid-glycolic acid copolymer or 2-hydroxybutyric acid-glycolic acid copolymer) used alone or in an admixture with polylactic acid. When glycolic acid copolymer is used in combination with polylactic acid, the ratio of glycolic acid copolymer/polylactic acid (weight %) can be, for example, about 10/90 to about 90/10. The preferred ratio is about 20/80 to about 80/20. The present invention also contemplates pure PGA carriers without added PLA.

In addition to the above mentioned biodegradable polymers, the present application also includes the use of poly(ethylene glycol)/poly(lactic-co-glycolic acid) copolymers and poly(caprolactone), i.e., PCL, which is another aliphatic polyester polymer that is much slower in degrading as compared with PLA or PLGA. Poly(ethylene glycol) or PEG is synonymous with poly(ethylene oxide) or PEO.

In another embodiment, encapsulant formulations including PEG-PLGA are contemplated. When these particles are placed in an aqueous environment, the PEG block of the polymeric material is much more hydrophilic and it orients itself to the outer shell of the particle; the PLGA block remains in the core. As is known to one skilled in the art, PEG inhibits protein binding and cellular recognition, making the particles ‘stealthy’ and reducing cellular uptake and phagocytosis. In yet another embodiment of the invention, PEG can be blended with a stabilizer such as, for example, poly(vinyl alcohol) and/or didodecyl dimethyl ammonium bromide (DMAB), which physically absorbs the polymer to the surface of the encapsulant.

Preferably, the encapsulating spheres have a size (i.e., diameter) of at least, about, or no more than, for example, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, or within a range bounded by any two of the foregoing values.

The degradation process of the above-described polymers, either in vivo or in vitro, is affected by several factors, including preparation method, molecular weight, composition, chemical structure, size, shape, crystallinity, surface morphology, hydrophobicity, glass transition temperature, site of active component implantation, physicochemical parameters in the surrounding environment (such as pH, temperature and ionic strength), and mechanism of hydrolysis. Specifically, the degradation behavior of the encapsulant depends on hydrophilicity of the polymer: the more hydrophilic the polymer, the more rapid its degradation. The hydrophilicity of the polymer is influenced by the ratio of crystalline to amorphous regions, which in turn is determined by copolymer composition and monomer stereochemistry. For example, PLGA copolymer prepared from L-PLA and PGA are crystalline copolymers, while those from D, L-PLA and PGA are amorphous in nature. Lactic acid, being more hydrophobic than glycolic acid, makes lactic acid-rich PLGA copolymers less hydrophilic, which slows down the degradation process.

In general, the degradation time will be shorter for lower molecular weight, more hydrophilic, more amorphous polymers and copolymers with higher content of glycolic acid. In accordance with these variables, the in vivo degradation rate of the D,L-PLGA copolymer may vary from a few weeks to more than 1 year.

By controlling the in vivo degradation rate of the encapsulant that carry the cABC enzyme, the release rate of the cABC enzyme can be adjusted or optimized. Preferably, the chondroitinase-loaded spheres release cABC for an extended period, e.g., ranging from about 1 week to about 1 year. More preferably, the loaded spheres release cABC at different rates, so that the overall release pattern of the cABC can be adapted for specific applications.

There are several common methods for the preparation of biodegradable spheres that can be used in the present invention. Some of these common methods include, for example, solvent-evaporation, salting-out, nanoprecipitation, and emulsification-diffusion. See, for example, Gurny et al., “Development of biodegradable and injectable latices for controlled release of potent drugs”, Drug Dev. Ind. Pharm., 7:1-25, 1981, Bindschaedler et al., “Process of preparing a powder of water-insoluble polymer which can be redispersed in a liquid phase, the resulting powder and utilization there of”, U.S. Pat. No. 4,468,350, 1990, Allèmann, et al., “Preparation of aqueous polymeric nanodispersions by a reversible salting-out process: influence of process parameters on particle size”, Int. J. Pharm., 87:247-253 1992; Fessi et al., “Procédé de préparation de systèmes colloïdaux dispersibles d'une substance, sous forme de nanoparticules”, French Patent, 2,608,988, 1998, Leroux, “New approach for the preparation of nanoparticles by an emulsification-diffusion method”, Eur. J. Pharm. Biopharm., 41:14-18, 1995, Gaspar et al., “Formulation of L-asparaginase-loaded poly(lactide-co-glycolide) nanoparticles: influence of polymer properties on enzyme loading, activity and in vitro release”, J. Control. Rel. 52:53-62, 1998; Wolf et al., “Stabilisation and determination of the biological activity of L-asparaginase in poly(D,L-lactide-co-glycolide) nanospheres”, Int. J. Pharm. 256:141-152, 2003, Kwon et al., “Preparation of PLGA nanoparticles containing estrogen by emulsification-diffusion method”, Coll. and Surf. A: Physicochem. and Eng. Asp. 182:123-130, 2001, Blanco and Alonso, “Development and characterization of protein-loaded poly(lactic/glycolic acid) nanospheres”, Eur. J. Pharm. Biopharm. 43:285-294, 1997, Vila et al., “Design of biodegradable particles for protein delivery”, J. Control. Rel. 78 15-24, 2002, Sahoo et al., “Residual polyvinyl alcohol associated with poly(D,L-lactide-co-glycolide) nanoparticles affects their physical properties and cellular uptake”, J. Control. Rel. 82:105-114, 2002, Song et al., “cAMP-induced switching in turning direction of nerve growth cones”, Nature 388: 275-279, 1997, Avgoustakis et al., “PLGA-mPEG nanoparticles of cisplatin: in vitro nanoparticle degradation, in vitro drug release and in vivo drug residence in blood properties”, J. Control. Rel. 79:123-135, 2002, and Labhasetwar et al., “Arterial uptake of biodegradable nanoparticles: effect of surface modifications”, J. Pharm. Sci. 87:1229-1234, (1998). The techniques disclosed in the aforementioned references for making the polymeric carriers are incorporated herein by reference.

In a particular embodiment, encapsulant spheres are prepared by a water-in-oil-in-water (w/o/w) solvent evaporation technique, such as described, for example, by Blanco et al., “Development and characterization of protein-loaded poly(lactic/glycolic acid) nanospheres”, Eur. J. Pharm. Biopharm. 43:285-294, (1997). In such a technique, one of the above mentioned polymeric materials, such as, for example, PLGA can be dissolved in an organic solvent, such as, ethyl acetate, to a concentration from about 2 to about 10 (w/v) and emulsified with an aqueous solution of reconsitituted cABC by probe sonication. The reconsitituted cABC (0.1 to 100 U/ml), which may optionally, but not necessarily, include BSA (Bovine Serum Albumin) or other carrier molecules as described in greater detail hereinafter, can be added in an amount from about 0.01 to about 1.0%, or in any other amount that would result in loaded particles that release cABC in concentrations sufficient to effectively reduce CSPG in the glial scar with little or no toxic effects and undesired inflammatory response, which can be readily determined by a person ordinarily skilled in the art through routine experimentation. These steps produce a water/oil emulsion (w/o), which can be further emulsified by sonication in an aqueous solution of an emulsifier such as PVA (1%, w/v) to form a second water/oil/water emulsion (w/o/w). The double emulsion can then be diluted into an aqueous solution of PVA and stirred for a time to evaporate the organic solvent. The encapsulant particles so formed can then be collected by known means including, for example, centrifugation, washed with distilled, deionized water, and then lyophilized using methods known to those skilled in the art.

Encapsulant particles of the present invention can also be prepared using the procedure disclosed, for example, in Quintanar-Guerrero et al., “Influence of stabilizing agents and preparative variables on the formation of poly(D,L-lactic acid) nanoparticles by an emulsification-diffusion technique”, Int. J. Pharm. 143:133-141, (1996) and Kwon et al., “Preparation of PLGA nanoparticles containing estrogen by emulsification-diffusion method”, Coll. and Surf. A: Physicochem. and Eng. Asp. 182:123-130, (2001). In such a method, one of the above mentioned polymeric materials, such as PLGA, is dissolved in an organic solvent to a concentration from about 1 to about 5% w/v, followed by the addition of reconstituted cABC, which may optionally, but not necessarily, include BSA or other carrier molecules. The amount of reconstituted cABC can be within the ranges mentioned above, or in any other range that would result in spheres that release cABC in concentrations sufficient to effectively remyelinate neurons with little or no toxic effects and undesired inflammatory response, which can be readily determined by a person ordinarily skilled in the art through routine experimentation. This organic phase mixture is then emulsified with an aqueous solution of an emulsifier such as PVA after mutual saturation of the two phases, using a high-speed homogenizer (8000 rpm).

Of the various techniques, it is preferred to use a solvent-evaporation technique or an emulsification-diffusion technique in fabricating the encapsulants that release active cABC. The emulsification-diffusion technique may offer some advantages over the solvent-evaporation technique, including, for example, an easier fabrication process, the potential for smaller encapsulant particles, and the ability to readily modify the surface of the polymeric matrix by using a stabilizer rather than having an additional surface modification step, as would be required in solvent evaporation.

A spontaneous emulsification method is also favored for preparation of the carriers described herein. In this method, a gradient in chemical potential is established at the interface of oil and water phases. As the co-solvent diffuses from the oil phase to the water phase, there is a longitudinal variation in surface tension and perturbation at the interface. The system is no longer in equilibrium and fluid movement persists, forming droplets. This spontaneous agitation is governed by the Marangoni effect. As organic solvent evaporates and surfactant stabilizes the droplets, particles form and harden. A film of polymer is deposited at the interface of the droplet and aqueous phase during hardening.

The methods described above can be readily used for preparation of spheres with sizes varying from a few nanometers to several hundred micrometers, by controlling the stirring rate and other processing conditions.

Optionally, the outer surface of the chondroitinase-loaded carriers is modified to carry a positive or negative charge. The charge can enhance adhesion of the spheres to an oppositely charged molecule prevalent at the demyelination site. In addition, surface charge can reduce aggregation of the spheres, thereby enhancing stability of the carrier formulation during storage.

Surface modification of the resultant spheres loaded with cABC can be made by treating the spheres with either a cationic surfactant or an anionic surfactant. The type of surfactant employed depends on the charge of the site being treated. The surfactants used in modifying the spheres of the present invention can be any pharmaceutical acceptable cationic or anionic surfactant. Cationic surfactants are used in forming positively charged encapsulant particles, which, in turn, are attracted by negatively charged sites. Some examples of cationic surfactants that can be employed in this invention include didodecyl dimethyl ammonium bromide and chitosan. Anionic surfactants are used in forming negatively charged encapsulant particles which, in turn, are attracted by positively charged sites. A particular anionic surfactant that can be employed in this invention includes polyvinyl alcohol.

The amount of surfactant used to modify the encapsulant surface may vary depending on the amount of encapsulant particles being treated. The surfactants can be admixed with water to provide an aqueous solution. In such an embodiment, the aqueous solution contains from about 1 to about 10% surfactant.

The surface modification occurs by contacting the encapsulant particles with the surfactant and providing a suspension by sonication over an ice bath. The suspension is then frozen over dry ice and lyophilized.

In order to obtain surface-modified encapsulant particles using the emulsification-diffusion technique, the organic phase will be emulsified with a cationic or anionic surfactant rather than PVA, under the conditions previously described. Following this step, water is added to the emulsion under magnetic stirring in order to allow for diffusion of organic solvent into the water and subsequent precipitation of the polymer. The organic solvent is then removed by filtration and the encapsulant particles are then lyophilized.

Preferably, the formulation of the present invention contains loaded spheres having a size smaller enough such that they are readily injectable in vivo, and large enough to remain extra-cellular and therefore induce a minimal inflammatory response when injected. In order to realize these advantages, the loaded spheres preferably have diameters ranging from about 100, 150, 200, or 250 nm to about 500, 600, 700, 800, 900, 1000, 1500, 2000, 2500, 3000, 3500, 4000, 4500, or 5000 nm.

Notwithstanding the technique used in forming the loaded spheres, the loading of the same with various concentrations of cABC can be achieved by using a stock solution of about 5 units/ml of cABC. The loading efficiency can be determined by at least two different methods. First, the washings from the preparation techniques are collected and analyzed for residual cABC content using SDS gel electrophoresis. The amount of loaded enzyme cABC is determined by the difference in the total amount of cABC added and the amount that is not incorporated, as measured by this technique. The loading efficiency is the ratio of enzyme incorporated to the total amount of cABC used in the fabrication process, expressed as a percentage.

The other method for evaluating enzyme loading efficiency is a direct assay of cABC content following an accelerated hydrolysis of the polymeric spheres. Samples of enzyme-loaded spheres are added to a 0.1 M sodium hydroxide (NaOH) solution containing 2% w/v sodium dodecyl sulfate (SDS) and shaken overnight. The solutions are neutralized with 1 M hydrochloric acid (HCl), diluted with distilled water, and filtered through a 0.2 μm Millipore membrane. The solutions are then analyzed by SDS gel electrophoresis and the enzyme loading efficiency determined as a function of fabrication technique and surface modification.

In one embodiment, the spheres containing cABC are administered alone in a treatment program. Alternatively, a treatment program can also include other therapeutic substances that promote healing and enhance nerve repair.

The chondroitinase-loaded spheres are preferably administered to a mammal at the site of demyelination by first suspending the loaded spheres in an aqueous vehicle and then injecting them through a hypodermic needle with a micropipette tip. Prior to injection, the loaded spheres can be sterilized with, preferably, gamma radiation or electron beam sterilization using techniques well known to those skilled in the art.

The dosage of chondroitinase may vary depending on the severity of demyelination. Typically, the dosage is from about 0.5 μl to about 100 μl, with a dosage from about 3 μl to about 20 μl being more typical. The above ranges are within therapeutic ranges which are capable of promoting healing, reducing scar formation and enhancing nerve repair at the site of the spinal cord injury.

The encapsulating particles are designed to degrade over time in an aqueous environment, resulting in the release of cABC. The time of release may vary depending on the type of polymeric materials used in forming the particles, as described hereinabove. A slow release formulation or an immediate formulation can be made by varying the polymeric composition and molecular weight. Typically, the release of the cABC and other substances from the particles is from about 24 hours to about 120 days. More preferably, the release of the cABC from the particles is from about 48 hours to about 30 days. The time-release property of the carriers can be readily measured in vitro by Bradford protein assays.

Examples have been set forth below for the purpose of illustration and to describe certain specific embodiments of the invention. However, the scope of this invention is not to be in any way limited by the examples set forth herein.

### EXAMPLE 1

### Materials and Methods

### Surgery

Spinal Contusion Injury: Female Long-Evans rats approximately 70 days old were used in this study. Rats were anesthetized via an intra-peritoneal (IP) injection of Sodium Pentobarbital (0.10 cc/0.10 kg). A laminectomy was first made at the T-9 vertebral level, and a moderate contusion injury was made using the NYU Impactor, by dropping a 10 g weighted rod on the exposed spinal cord from a height of 25 mm.

Saline or cABC Injections: Immediately following impaction, two injections (3 μL each) of Saline or 20 mU cABC were made using a Hamilton syringe in a stereotaxic apparatus at the rostral and caudal aspect of the lesion.

BrdU Injections: Prior to sacrificing, animals received 2 injections of BrdU (IP; 50 μg/gm body weight) 24 hours apart.

### Histology

Tissue Harvest: At different survival times, (1-wk, 2-wks, and 1-mo. post-SCI; N=3) animals were euthanized with 0.5 cc Fatal Plus and then received a direct cardiac injection of 0.2 cc Heparin (100 U/ml) and 0.2 cc Lidocaine (2%), followed by transcardial perfusion with 500 ml 0.2M PBS, followed by 4% Paraformaldehyde (PFA; pH 7.4). Spinal cord were dissected out and post-fixed in 4% PFA for 1 week, followed by cryopreservation treatment in 20% Sucrose in 1× PBS. Longitudinal frozen sections were cut (20 μm), and mounted on slides and stored at −20° C. until processing.

Immunohistochemistry: Tissue segments containing the lesion site were probed using 2 markers for OPCs: anti-PDGFRα (1:250; Santa Cruz) and anti-Olig1 (1:250; Millipore). In adjacent tissue segments, tissue was probed for actively dividing OPC using anti-Olig1 (1:250; Millipore) and anti-BrdU (1:200; Abcam). Tissue was blocked for 1 hr prior to addition of the primary antibodies. Tissue segments were incubated in primary antibodies overnight at 4° C. The following day, primary antibodies were detected using goat-anti-Rabbit/Mouse-Alexa Fluors 488 or 555.

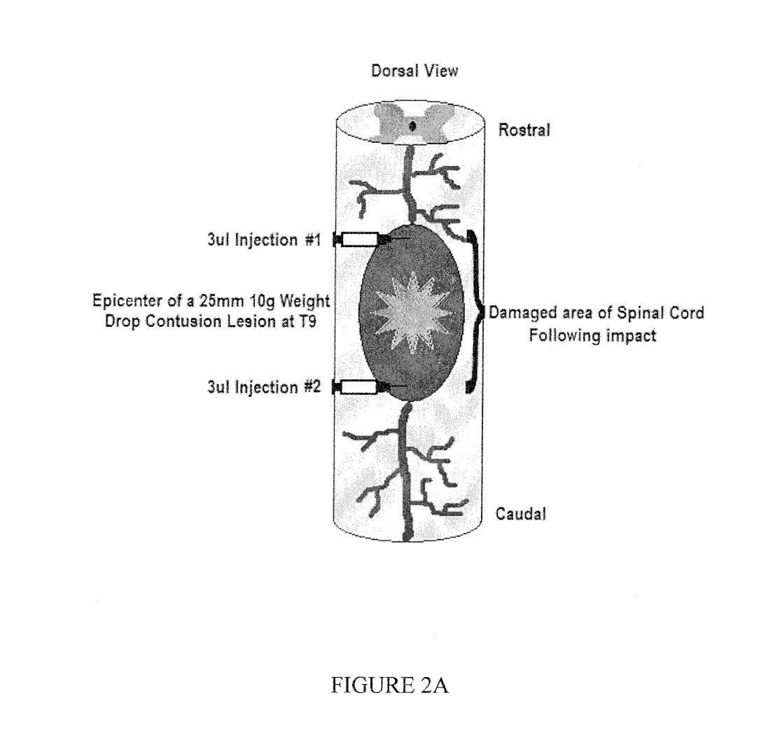

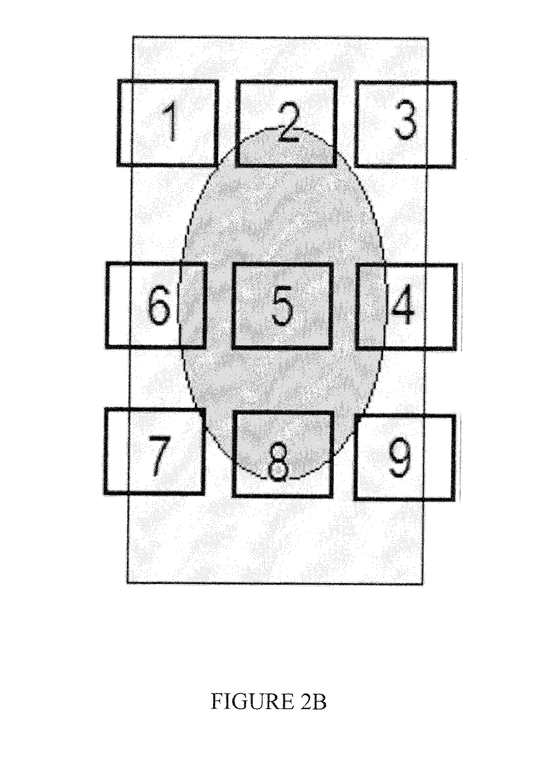

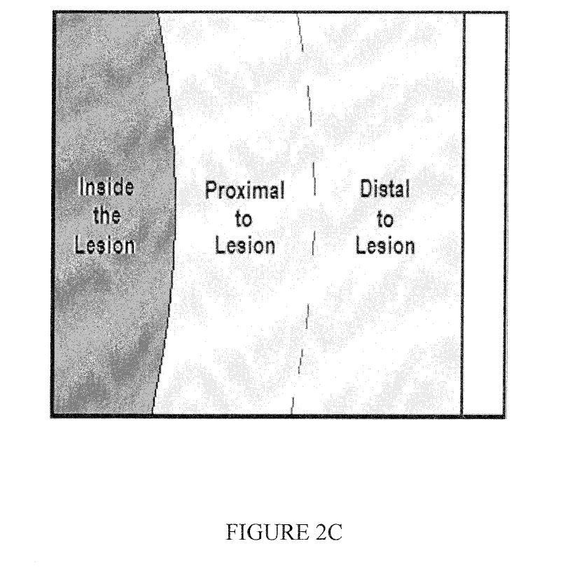

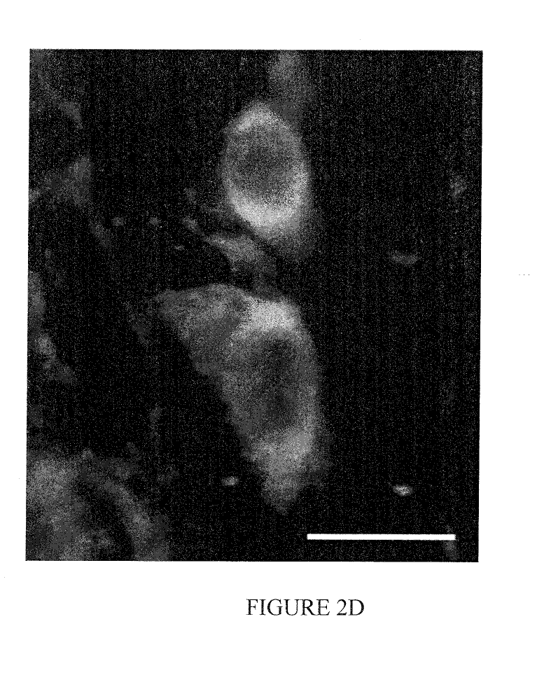

Data Analysis: To determine if cABC treatment, and subsequent digestion of the CSPGs would have any effect on the abilities of OPCs to infiltrate the lesion site, we quantified the number of double labeled Olig1+ and PDGFRα+OPCs, found in the tissue adjacent to the lesion site. To quantify the number of OPCs, nine micrographs were taken of a single tissue section, in the area indicated in FIG. 2. Two scorers counted the number of Olig1+ and PDGFRα+ cell profiles that were observed in each section. All micrographs were blinded to assure the scorers could not tell what condition as they counted cells.

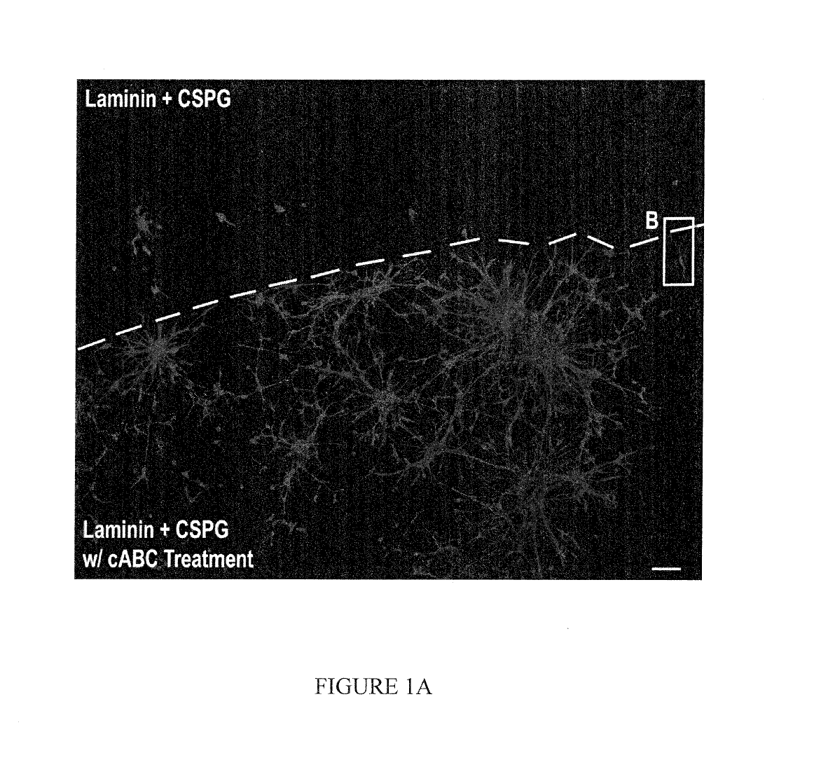

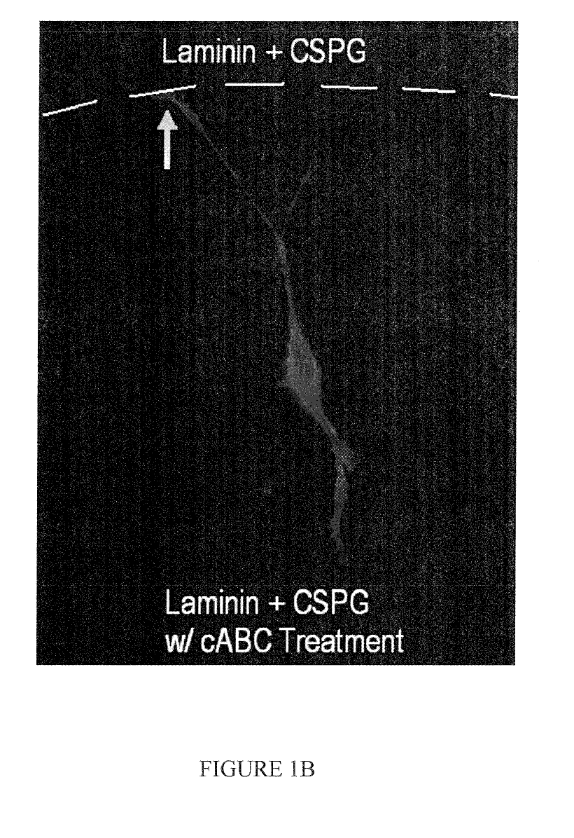

OPCs Actively Avoid Areas of CSPGs. Primary OPC cells were plated on 12 mm coverslips coated with laminin and 50 μg/ml CSPGs. Immediately prior to plating, one-half of the coverslip was digested with the enzyme cABC for 30 minutes. OPC cells were initially plated in the middle of the coverslip, and maintained in media with mitogens to prevent their differentiation. FIG. 1A is a micrograph of cells, treated as above, fixed with 4% paraformaldehyde 96 hours post-plating. The border between cABC treated and untreated is clearly delineated by OPC location and growth. Most OPC stayed on the digested section of the coverslip. Those that attached to the undigested CSPG show very little process outgrowth. FIG. 1B shows the cells under higher magnification. In the magnified slide, processes extending from OPC can be observed contacting the untreated border and abruptly turning away.

In vivo Experimental and Data Analysis Schematics. FIGS. 2A-F are representations illustrating the experimental injury and treatment paradigm, and the methodology used for quantifying OPC numbers. FIG. 2A is a drawing depicting a preferred methodology wherein animals received a 10 gram weight drop contusion injury from a height of 25 mm, and immediately following injury, were administered two injections of either saline or 20 mU cABC. FIG. 2B is a drawing depicting a preferred methodology wherein, to determine the effectiveness of cABC treatment following SCI, 9 photomicrographs were taken in the general areas indicated. All photomicrographs were taken at 20×, to provide the maximal amount of area, while still being able to visualize a clear cell morphology. FIG. 2C is a drawing depicting a preferred methodology, wherein, during data quantification, the geographic distribution of OPCs was determined by dividing all the micrographs (except area #5) into the three zones indicated. FIG. 2D is a micrograph showing that only cells that were double stained for both PDGFRα and Olig1 and displayed a clear cell morphology were counted in the analysis (scale bar=10 μm).

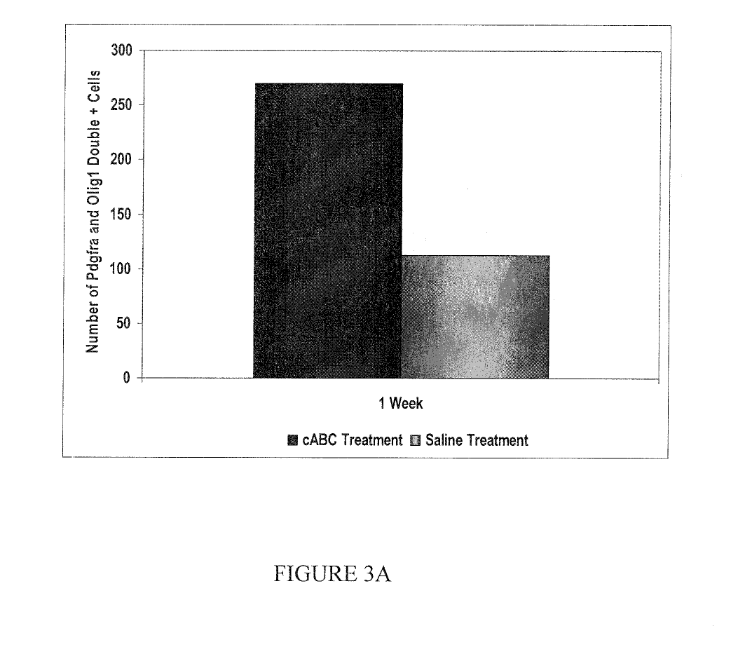

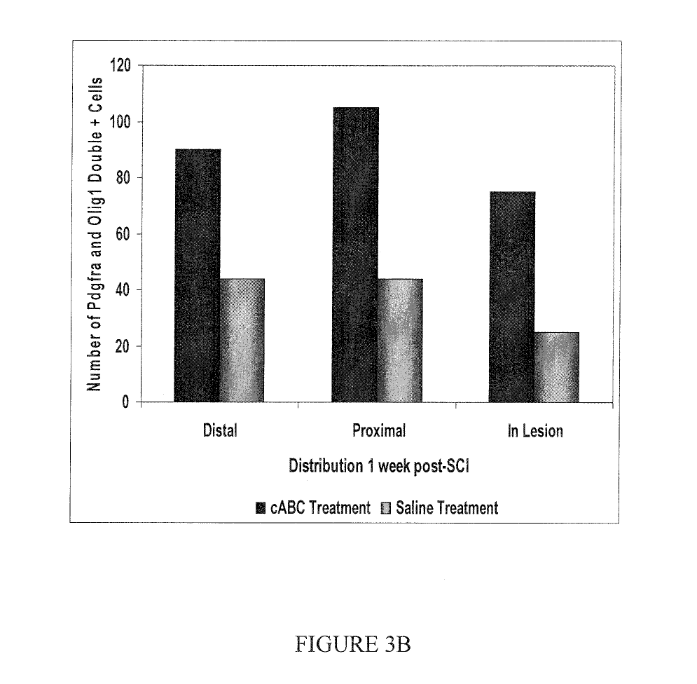

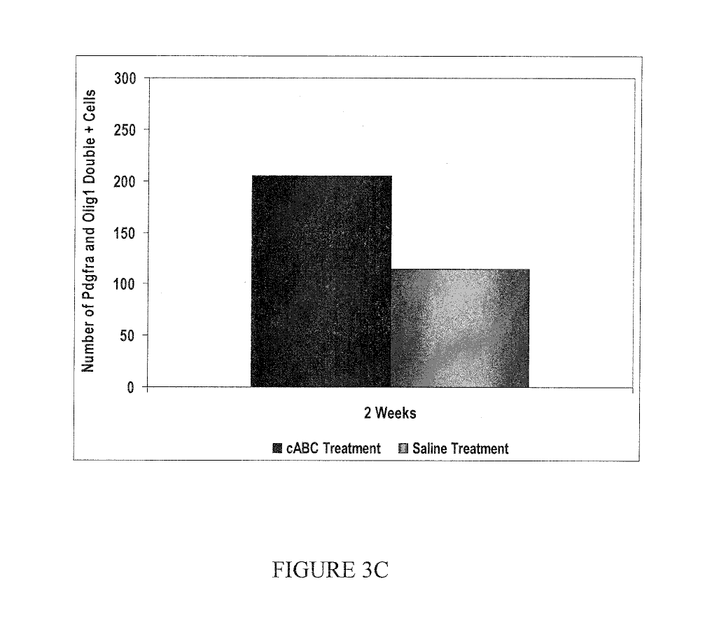

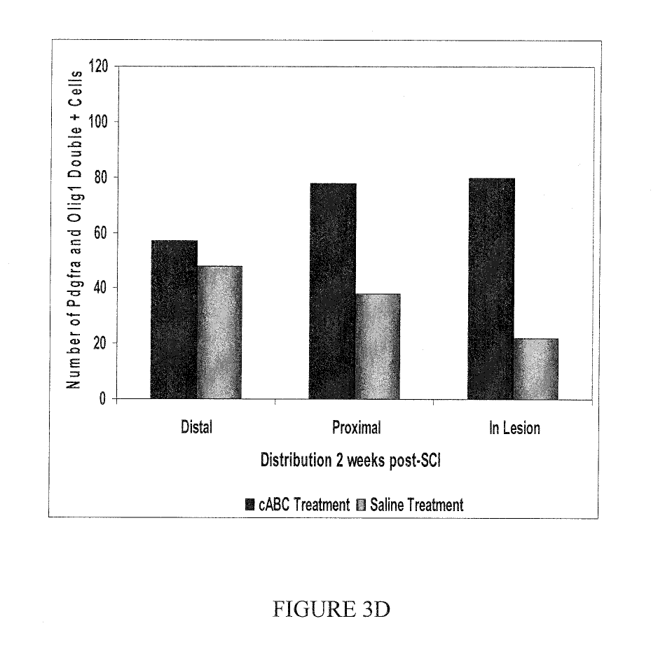

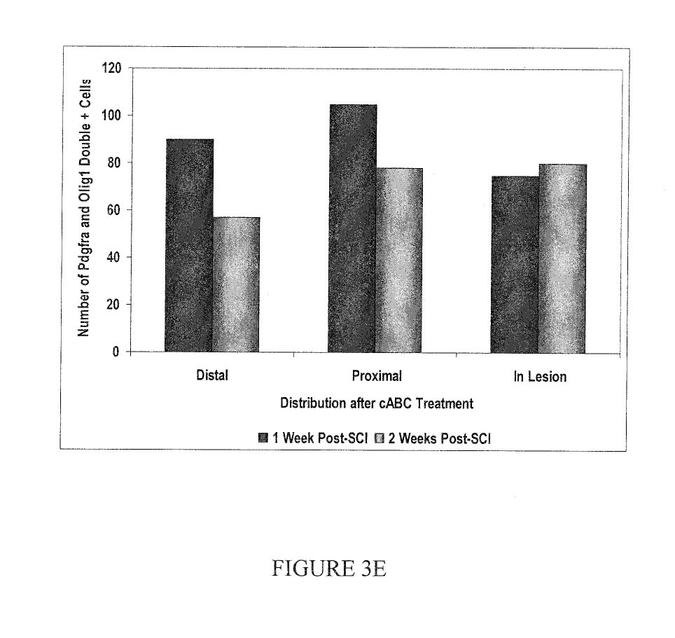

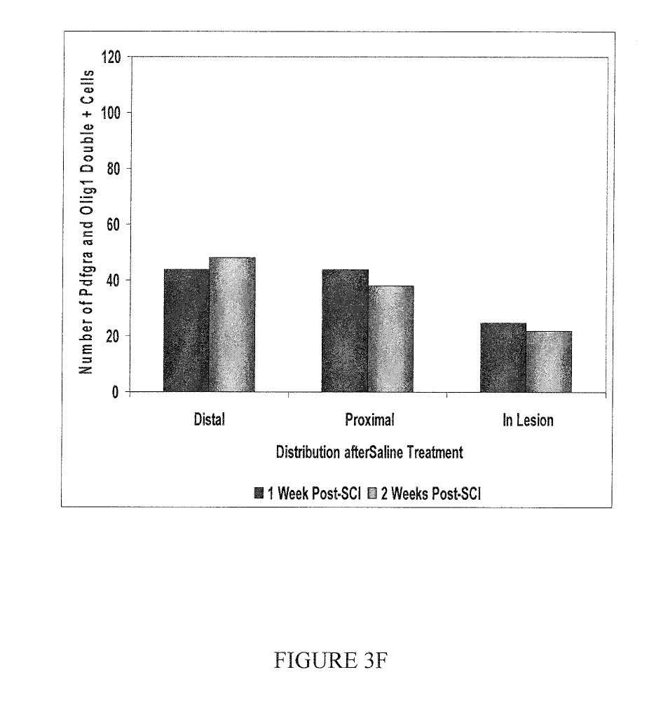

cABC Treatment Promotes Greater OPC Migration After SCI. As shown by FIGS. 3A-F, the use of cABC resulted in a doubling in the number of PDGFRα/Olig1 labeled OPCs at both 1 week (Fig. A) and 2 weeks (Fig. C). Moreover, an examination of the tissue distribution of OPCs at both at one week (Fig. B) and two weeks (Fig. D) post-SCI, show that most OPCs were located either proximal to the lesion or within the lesion site itself. This is in contrast to the control animals, where the majority of the OPCs were found in the lateral spared rim of white matter or the zone distal to the lesion cavity at both one week (Fig. B) and two weeks (Fig. D). It appears that cABC digestion allows for enhanced migration of OPCs throughout the lesioned area, as the peak location of OPCs changes over time. At one week, the greatest number of OPCs is observed in the area just proximal to the lesion (Fig. E), but at two weeks the majority of cells are in the lesion itself. There is no such shift in the saline controls, as the peak number of cells remain in the spared rim of tissue distal to the lesion (Fig. F). These results suggest that the digestion of CSPGs with cABC creates an environment that allows for a rapid and unrestricted infiltration of OPCs after SCI.

The loss of motor function following a spinal cord injury (SCI) can be attributed, in part, to the demyelination of intact axons in and around the injury site. The initial trauma destroys many oligodendrocytes (OL), the myelinating cell in the spinal cord, and some studies suggests that OL loss continues for long times post-injury. Endogenous oligodendrocyte progenitor cells (OPCs) that persist near the lesion site are unable to effectively remyelinate spared axons. One reason for this may be the glial scar formed after SCI, which acts as both a physical and chemical barrier to regeneration. The glial scar is enriched in chondroitin sulfate proteoglycans (CSPGs), which strongly inhibit axonal sprouting and regrowth. Recent findings suggest that the expression of CSPGs may also inhibit remyelination. Our in vitro studies demonstrated that differentiation of OPCs is inhibited in the presence of CSPGs, particularly those that are up-regulated post-SCI. In addition, when OPCs come in contact with a CSPG rich area, their processes retract and turn away from the CSPG deposits. These results suggest the glial scar not only inhibits axonal regeneration, but remyelination as well.

Treatment with the enzyme chondroitinase ABC (cABC) enhances axonal sprouting post-SCI by digesting the glycosaminoglycan (GAG) side chains from the core protein of CSPGs. In this study, the effects of cABC treatment on endogenous OPC migration in vivo after a spinal contusion injury were examined. The total number of OPCs surrounding the lesion site was greatly enhanced after cABC treatment compared to controls. Moreover, there was a noticeable difference in the distribution of OPCs. It was evident that after cABC digestion, most OPCs migrated proximally or directly into the lesion. In contrast, there were lower numbers of OPCs in control animals, and the majority were located in the lateral rim of spared tissue. These data suggest that cABC treatment can create a permissive environment that allows for regenerative sprouting, and OPC infiltration after SCI. Chondroitinase treatment has been shown to promote greater migration of OPCs following spinal cord injury. These progenitors are the cells that eventually remyelinate axons.

While there have been shown and described what are at present considered the preferred embodiments of the invention, those skilled in the art may make various changes and modifications which remain within the scope of the invention defined by the appended claims.

What is claimed is:

1. A method of treating a mammal suffering from a demyelinating disease or condition, the method comprising administering chondroitinase to said mammal at a site where demyelination has occurred.

2. The method of claim 1, wherein said demyelinating disease or condition is an idiopathic inflammatory demyelinating disease.

3. The method of claim 1, wherein said demyelinating disease or condition is a form of multiple sclerosis.

4. The method of claim 1, wherein said demyelinating disease or condition is a spinal cord injury.

5. The method of claim 4, wherein said spinal cord injury is acute or chronic.

6. The method of claim 1, wherein the administering is by injection of a sterile solution comprising chondroitinase and a suitable vehicle in which chondroitinase is dissolved.

7. The method of claim 1, wherein the chondroitinase is in the form of biodegradable spheres comprising a biodegradable encapsulant and chondroitinase encapsulated therein.

8. The method of claim 7, wherein said biodegradable spheres have a particle size of 250 nm to 2 μm.

9. The method of claim 7, wherein said biodegradable spheres have a particle size of 250 nm to 1 μm.

10. The method of claim 7, wherein said biodegradable encapsulant is comprised of a biodegradable polymer or polymer capable of releasing the chondroitinase into a site at which the biodegradable spheres are administered.

11. The method of claim 10, wherein said biodegradable polymer or copolymer comprises an aliphatic polyester derived from one or more building blocks selected from the group consisting of α-hydroxycarboxylic acids, hydroxydicarboxylic acids, hydroxytricarboxylic acids, poly-α-cyanoacrylic esters, and amino acids.

12. The method of claim 10, wherein said biodegradable polymer or copolymer is derived from one or more kinds of α-hydroxycarboxylic acids.

13. The method of claim 12, wherein said one or more kinds of α-hydroxycarboxylic acids comprise glycolic acid and lactic acid.

14. The method of claim 1, wherein said biodegradable spheres comprise polylactic acid, polyglycolic acid, or a copolymer of PLA and PGA.

15. The method of claim 1, wherein said chondroitinase is administered simultaneously with a neurotrophin.

16. The method of claim 7, wherein said biodegradable spheres further comprise a neurotrophin encapsulated therein.

17. The method of claim 1, wherein said chondroitinase is administered simultaneously with one or more carrier proteins selected from the group consisting of Bovine Serum Albumin (BSA), Keyhole Limpet Hemocyanin (KLH), Ovalbumin (OVA), Fetal Bovine Serum (FBS), Thyroglobulin (THY), and Human Serum Albumin (HSA).

18. The method of claim 7, wherein said biodegradable spheres further comprise, encapsulated therein, one or more carrier proteins selected from the group consisting of Bovine Serum Albumin (BSA), Keyhole Limpet Hemocyanin (KLH), Ovalbumin (OVA), Fetal Bovine Serum (FBS), Thyroglobulin (THY), and Human Serum Albumin (HSA).
